(19) United States
(12) Patent Application Publication
(10) Pub. No.: US 2017/0224874 A1
(43) Pub. Date: Aug. 10, 2017

(54) HYDROGELS FOR TREATING AND AMELIORATING WOUNDS AND METHODS FOR MAKING AND USING THEM

(71) Applicant: Vicus Therapeutics, LLC, Morristown, NJ (US)

(72) Inventors: John Maki, Mendham, NJ (US); Newell Bascomb, Hendersonville, NC (US); Fredric Young, Los Altos, CA (US)

(21) Appl. No.: 15/320,731

(22) PCT Filed: Jul. 1, 2015
(86) PCT No.: PCT/US15/38848

§ 371 Date: Dec. 20, 2016

Related U.S. Application Data
(60) Provisional application No. 62/019,786, filed on Jul. 1, 2014.
(60) Provisional application No. 62/042,776, filed on Aug. 27, 2014.

(51) Int. Cl. A61L 27/60; A61F 2/10; A61L 27/36; A61L 27/56; A61L 27/52; A61L 27/54

(52) U.S. Cl. CPC A61L 27/60; A61L 27/52; A61L 27/54; A61L 27/3691; A61L 27/3687; A61L 27/56; A61L 27/3641; A61L 27/362; A61F 2/105; A61L 2430/34; A61L 2300/25

(57) ABSTRACT

In alternative embodiments, provided are compositions, e.g., pharmaceutical compositions, formulations, kits and other products of manufacture, comprising a hydrogel and active ingredients, including mixed thickness skin micrografts, or full or split-thickness skin grafts, contained or mixed in or within the hydrogel; and methods for making and using them. In alternative embodiments, compositions and methods as provided herein are used for the treatment or amelioration of wounds and surgical sites, and include compositions and methods for micrografting, or for micrografting a wound, or for micrografting a wound for rapid re-epithelialization, or for micrografting a wound for rapid re-epithelialization of large non-healing wounds.

### RELATED APPLICATIONS

This Patent Convention Treaty (PCT) International Application claims the benefit of priority under 35 U.S.C. §119(e) of U.S. Provisional Application Ser. No. (U.S. Ser. No.) 62/019,786, filed Jul. 01, 2014, and U.S. Ser. No. 62/042,776, filed Aug. 27, 2014. The aforementioned applications are expressly incorporated herein by reference their entirety and for all purposes.

### FIELD OF THE INVENTION

This invention relates generally to medicine, pharmaceutical formulations and medical devices. In alternative embodiments, provided are compositions, e.g., pharmaceutical compositions, formulations, kits and other products of manufacture, comprising a hydrogel and active ingredients, including mixed thickness skin micrografts, or full or split-thickness skin grafts, contained or mixed in or within the hydrogel; and methods for making and using them. In alternative embodiments, compositions and methods as provided herein are used for the treatment or amelioration of wounds and surgical sites, and include compositions and methods for micrografting, or for micrografting a wound, or for micrografting a wound for rapid re-epithelialization, or for micrografting a wound for rapid re-epithelialization of large non-healing wounds.

### BACKGROUND

Non-healing or chronic wounds can shows little or no improvement after four weeks or does not heal in eight weeks, and can pose the risk of infection, which can lead to a more serious conditions, possibly resulting in the loss of a limb. Individuals having diabetes, circulatory problems, compromised immune system, severe burns or genetic blistering diseases, including epidermolysis bullosa are at risk for having non-healing or chronic wounds and their more severe sequelae. Living with a chronic wound can have a significant impact on both the physical and psychological health of an individual; patients may suffer from multiple effects including reduced mobility, pain, poor nutrition and depression. Chronic wounds mostly affect people over the age of 60, and the incidence is 0.78% of the population and the prevalence ranges from 0.18 to 0.32%. As the population ages, the number of chronic wounds is expected to rise.

### SUMMARY

In alternative embodiments, provided are products of manufacture (articles of manufacture), devices (e.g., medical devices) or compositions, comprising:

(a) a sterile hydrogel comprising a hydrogel material, wherein the hydrogel is:
(i) in a substantially liquid form capable of setting, gelling or self-assembling;
(ii) a partially assembled or gelled hydrogel, in a partially assembled or gelled form; or,
(iii) in a set, gelled or self-assembled state; or a substantially set, gelled or self-assembled state,
and optionally the set, gelled or self-assembled state is in situ; and

(b)

(1)

(i) a mixed thickness skin micrograft, a split-thickness skin graft, or a full thickness skin graft,

wherein the mixed thickness skin micrograft, split-thickness skin graft, or full thickness skin graft is dispersed in, or mixed into, or substantially evenly distributed throughout, the sterile hydrogel,

and optionally the mixed thickness skin micrograft, split-thickness skin graft, or full thickness skin graft further comprises, or is dispersed in, or mixed into, or substantially evenly distributed throughout, a sterile pure water or a sterile isotonic solution or buffer, or equivalent,

and optionally the micrograft or skin graft is an autologous micrograft;

(ii) a skin tissue column, a microscopic skin tissue column or a skin graft comprising a full-thickness column of skin tissue, a “fractional skin harvesting (FSH)” graft, an ultra-micrograft, a microscopic skin tissue column (MSTC),

wherein optionally the skin tissue column, the micrograft, FSH or skin graft is an autologous graft,

and optionally the skin tissue column, the micrograft, FSH or skin graft is derived from revertant Epidermolysis Bullosa (EB) skin tissue;

(iii) a cell, a tissue or an organ preparation,

wherein optionally the tissue or organ is partially, substantially or fully dissociated or disrupted, and optionally the partially, substantially or fully dissociated or disrupted tissue or organ is dispersed in, or mixed into, or substantially evenly distributed throughout, the sterile hydrogel, and optionally the partially, substantially or fully dissociated or disrupted tissue or organ is substantially evenly distributed throughout, a sterile pure water or a sterile isotonic solution or buffer, or equivalent,

and optionally the tissue or organ is partially, substantially or fully dissociated or disrupted by an enzymatic treatment or a physical dissociation or disruption,

and optionally the enzymatic treatment comprises a collagenase treatment,

and optionally the cell, tissue or organ preparation comprises a collagenase, and optionally the collagenase treatment comprises use of about 600U collagenase/ml tissue,

and optionally the cell is a stem cell, or optionally the cell is derived from a bone, a cartilage, a tendon, a muscle, a bladder, a nervous tissue, a spinal nerve tissue, a brain tissue, a glial tissue, an eye, a skin tissue, a venous or arterial tissue, a mucosal tissue, a urothelial mucosal tissue, a muscle or heart tissue, a bladder, a brain, a heart, a liver, a pancreas, or a urethra,

and optionally the cell is a stem cell, an undifferentiated cell, a de-differentiated cell, a pluripotent cell, an omnipotent cell, an umbilical cord blood cell, or a tissue culture cell,

and optionally the organ is a bladder, a brain, a heart, a muscle, a bone, a tendon, a cartilage, a liver, a pancreas, a urethra or an eye, or

(iv) the mixed thickness skin micrograft, a split-thickness skin graft, or a full thickness skin graft of (i), the skin tissue column, microscopic skin tissue column or skin graft comprising a full-thickness column of skin tissue of (ii), or the cell, a tissue or an organ preparation of (iii), further comprising an enzyme, wherein optionally the enzyme is a collagenase or a hyaluronidase;

(2) a hemostatic agent, wherein optionally the hemostatic agent comprises a tranexamic acid (trans-4-(aminomethyl)cyclohexanecarboxylic acid), or a synthetic analog of the amino acid lysine;

(3) a growth factor or an accelerator of cell migration,

wherein optionally the growth factor is an erythropoietin, a recombinant erythropoietin, or an epoetin alfa (e.g., a PROCRIT™ or an EPOGEN™); a granulocyte colony-stimulating factor (G-CSF or GCSF), also known as colony-stimulating factor 3 (CSF 3); a filgrastin or a G-CSF analog, or a FILCAD™ (Cadila Pharmaceuticals), IMUMAX™ (Abbott Laboratories), GRAFEEL™ (Dr. Reddy's Laboratories), NEUKINE (Intas Biopharmaceuticals), EMGRAST™ (Emcure Pharmaceuticals), RELIGRAST™ (Reliance Life Sciences), ZARZIO™ (Sandoz), or a NUFIL™ (Biocon); a keratinocyte growth factor or a palifermin or a KEPIVANCE™ (Biovitrum); a gamma-aminobutyric acid (GABA); or, any combination thereof,

wherein optionally the accelerator of cell migration comprises an inhibitor of a microtubule-severing enzyme, an inhibitor of microtubule degradation or an accelerator of microtubule formation, and optionally the microtubule-severing enzyme comprises an fidgetin-like 2 (FL2) enzyme and the inhibitor of a microtubule-severing enzyme comprises an inhibitor of FL2, and optionally the FL2 inhibitor comprises an FL2-inhibiting antisense nucleotide (e.g., an antisense RNA) or an FL2-inhibiting siRNA;

(4) an anti-oxidant,

wherein optionally the anti-oxidant comprises: a glycyrrhetinic acid (GA) (also known as enoxolone), a nicotinamide (also known as vitamin B3), a niacin, a vitamin A, a vitamin C (ascorbate), a vitamin E or any tocopherol or tocotrienol, or a deferoxamine (also known as desferrioxamine B, desferoxamine B, DFO, DFO-B, DFOA, DFB or desferal),

and optionally the deferoxamine is at a concentration of about 0.1%,

and optionally the nicotinamide is at a concentration of about 0.1%;

(5) an aminoglycoside, wherein optionally the aminoglycoside comprises a gentamicin; or

(6) a combination of (1), (2), (3) and (4);

a combination of (2), (3) and (4);

a combination of (2) and (3);

a combination of (3) and (4);

a combination of (2) and (4);

a combination of (1), (2) and (3);

a combination of (1), (2) and (4);

a combination of (1) , (3) and (4);

a combination of (1) and (2);

a combination of (1) and (3);

a combination of (1) and (4);

a combination of (1), (2), (3), (4) and (5);

a combination of (1), and (5);

a combination of (1), (2), and (5);

a combination of (1), (3) and (5);

a combination of (1), (4) and (5);

a combination of (1), (2), (3) and (5);

a combination of (1), (2), (4) and (5);

a combination of (1), (3), (4) and (5); or

any combination of (1), (2), (3), (4) and/or (5).

In alternative embodiments, of the products of manufacture, devices, or compositions as provided herein:

(a) the mixed thickness skin micrograft, split-thickness skin graft, or full thickness skin graft of is a minced mixed thickness skin micrograft, split-thickness skin graft, or full thickness skin graft;

(b) the mixed thickness skin micrograft, split-thickness skin graft, full thickness skin graft, mixed thickness skin micrograft, skin tissue column, microscopic skin tissue column, “fractional skin harvesting (FSH)” graft, ultra-micrograft, or a microscopic skin tissue column (MSTC), has been:

(i) subjected to a mincing procedure,

(ii) substantially cut into a plurality of pieces,

(iii) subjected to a collagenase treatment, and optionally the collagenase treatment comprises use of about 600U collagenase/ml tissue, or

(iv) has been subjected to a mincing procedure or substantially cut into a plurality of pieces and subjected to a collagenase treatment;

and optionally the mixed thickness skin micrograft, split-thickness skin graft, or full thickness skin graft is minced before mixing a sterile pure water or isotonic solution or buffer with the mixed thickness skin micrograft, split-thickness skin graft, or full thickness skin graft,

and optionally the mixed thickness skin micrograft, split-thickness skin graft, or full thickness skin graft is harvested and/or minced using an XPANSION® device or an XPANSION MICROGRAFTING SYSTEM° (SteadMed Medical, Fort Worth, Tex.);

(c) the mixed thickness skin micrograft, split-thickness skin graft, or full thickness skin graft is harvested from a host or donor as a graft of about 0.012″ to 0.016″ in thickness,

wherein optionally the harvested mixed thickness skin micrograft, split-thickness skin graft, or full thickness skin graft is mixed in sufficient pure water, isotonic solution or buffer to result in a suspension,

and optionally the amount of mixed thickness skin micrograft, split-thickness skin graft, or full thickness skin graft is sufficient to substantially cover a donor site tissue area equal to: about ⅓^{rd} to 1/100^{th} , or about 1/10^{th} to 1/100^{th}, of the area of an intended recipient site (area to receive the graft); or

(d) the mixed thickness skin micrograft, split-thickness skin graft, or full thickness skin graft is a human mixed thickness skin micrograft, split-thickness skin graft, or full thickness skin graft.

In alternative embodiments, of the products of manufacture, devices, or compositions as provided herein:

(a) the hydrogel is capable of self-assembling, gelling or setting when exposed to an environment comprising a salt concentrations ≧1 mM (or gelation, self-assembly or setting is initiated by salt concentrations ≧1 mM);

(b) the hydrogel is capable of self-assembling, gelling or setting into a 3D hydrogel having a nanometer scale and/or a fibrous structure with an average pore size of between about 50 to 200 nm; or

(c) the hydrogel is at a concentration of about: 0.1% to 5% (w/v), 0.5% to 4% (w/v), 1% to 3% (w/v), 1% to 10% (w/v), 1% to 15% (w/v), 1% to 20% (w/v), 1% to 25% (w/v), 1% to 30% (w/v), 1% to 40% (w/v), or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more (w/v); or

(d) the product of manufacture, device or composition of (a), wherein:
(1) the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
(2) the PBS is at a concentration of about 0.25% to 2.5%, 0.25% to 1.5%, 0.5% to 1.0%, 0.54%, 0.6%, 0.7%, 0.8%, 0.9% or 1.0%,or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more.

In alternative embodiments, of the products of manufacture, devices, or compositions as provided herein:

(a) the hydrogel or hydrogel material comprises a self-assembling peptide;

(b) the hydrogel or hydrogel material comprises a plurality of synthetic peptides characterized by stable B-sheet structure with ionic side-chain interactions after setting, gelling or self-assembling;

(c) the hydrogel or hydrogel material comprises a 16-amino acid synthetic peptide (Ac-[RADA]₄-CONH₂), or SEQ ID NO:1, and optionally the hydrogel comprises PURAMATRIX™ (PuraMatrix™) (BD Biosciences, San Jose, Calif.), or PURADERM™ (PuraDerm™) (3DMatrix, Ltd, Tokyo, Japan);

(d) the hydrogel or hydrogel material comprises a self-assembling peptide comprising the sequence Lys-Leu-Asp-Leu-Lys-Leu-Asp-Leu-Lys-Leu-Asp-Leu (KLDL)₃ (SEQ ID NO:2);

(e) the hydrogel or hydrogel material comprises a self-assembling peptide comprising the sequence Ile-Glu-Ile-Lys-Ile-Glu-Ile-Lys-Ile-Glu-Ile-Lys-Ile (IEIK)₃I (SEQ ID NO:3);

(f) the hydrogel or hydrogel material comprises a cellulose, a chitin, a chitosan or a deacetylated chitin, a laminin, a collagen, an elastin, a fibrin, a gelatin, an alginic acid, a hyaluronic acid (HA), or a combination thereof,

wherein optionally the HA comprise a thiolated HA or a tyraminated HA,

or optionally the collagen comprises a collagen IV or a collagen I,

or optionally the cellulose comprises a hemicellulose methyl cellulose (MC), a hydroxypropyl cellulose (HPC), a hydroxypropylmethyl cellulose (HPMC), a carboxymethyl cellulose (CMC) or a cellulose-inorganic hybrid hydrogel;

(g) the hydrogel or hydrogel material comprises a polyethylene glycol (PEG), a polyethelene glycol diacrylate (PEGDA), an ethylene glycol dimethacrylate (EGDMA); a cyclodextrin; a p-dioxanone; a hydroxyethyl methacrylate; a poly(methyl methacrylate); a methylene-bis-acrylamide; a poly(acrylic acid); a polyacrylonitrile; a poly(butylene oxide); a polycaprolactone; a poly(ethylene imine); a poly(ethylene oxide); a poly(ethyl methacrylate); a propylene fumarate; a poly(glucosylethyl methacrylate); a poly(hydroxy butyrate); a poly(hydroxyethyl methacrylate); a poly(hydroxypropyl methacrylamide); a poly(lactic acid); a poly(lactic-co-glycolic acid); PNIPAAm, poly(N-isopropyl acrylamide); a poly(N-vinyl pyrrolidone); a poly(propylene oxide); a poly(vinyl alcohol); a poly(vinyl acetate); a poly(vinyl amine), or any combination thereof; or

(h) the hydrogel or hydrogel material comprises any combination of (a) to (g).

In alternative embodiments, of the products of manufacture, devices, or compositions as provided herein:

(a) the isotonic solution or buffer comprises a saline, a phosphate buffered saline (PBS), or an equivalent buffer;

(b) the product of manufacture, device or composition of (a), wherein:
(1) the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
(2) the PBS is at a concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or

(c) the mixed thickness skin micrograft, split-thickness skin graft, full thickness skin graft, mixed thickness skin micrograft, skin tissue column, microscopic skin tissue column, “fractional skin harvesting (FSH)” graft, ultra-micrograft, or a microscopic skin tissue column (MSTC), is mixed with sufficient pure water, isotonic solution or buffer to result in a suspension comprising between about a 1.5% to 15% concentration (skin micrograft) per unit weight, or between about a 1.0% to 20% concentration (skin micrograft) per unit weight, or between about a 1.0% to 20% concentration (skin micrograft) per unit volume, or between about a 0.1% to 10% concentration (skin micrograft) per unit volume, of mixed thickness skin micrograft, split-thickness skin graft, or full thickness skin graft in the pure water, isotonic solution or buffer,

and optionally the suspension is then mixed at a ratio of 2 parts suspension to 3 parts hydrogel solution, or 1 to 3 parts suspension to 2 to 4 parts hydrogel solution, to make final product of manufacture or composition having:
about 0.25% to 3.0%, 0.5% to 2.0%, 1%, 1.5%, 2%, 2.5%, 3% or more hydrogel,
about 1% to 10% mixed thickness skin micrograft, split-thickness skin graft, or full thickness skin graft suspension; and/or
about 0.25% to 1.5%, 0.5% to 1.0%, 0.54%, 0.6%, 0.7%, 0.8%, 0.9% or 1.0% or more pure water, isotonic solution or buffer.

In alternative embodiments, of the products of manufacture, devices, or compositions as provided herein, the product of manufacture or composition, or the hydrogel, or isotonic solution, comprises:

(a) a tranexamic acid or a synthetic analog of the amino acid lysine;

(b) an erythropoietin, a recombinant erythropoietin, or an epoetin alfa (e.g., PROCRIT™ or EPOGEN™); and

(c) an anti-oxidant, wherein optionally the anti-oxidant comprises: a glycyrrhetinic acid (GA) (also known as enoxolone), a nicotinamide (also known as vitamin B3), a niacin, a vitamin A, a vitamin C, a vitamin E, or a deferoxamine (also known as desferrioxamine B, desferoxamine B, DFO, DFO-B, DFOA, DFB or desferal), and optionally the deferoxamine is at a concentration of about 0.1%, and optionally the nicotinamide is at a concentration of about 0.1%.

In alternative embodiments, of the products of manufacture, devices, or compositions as provided herein: (a) the product of manufacture, device or composition is in situ; or (b) the product of manufacture or device is or comprises a component or part of a medical device.

In alternative embodiments, provided are kits, or integrated point of care mixing kits, comprising a product of manufacture, a device, or a composition as provided herein.

In alternative embodiments, provided are methods for treating and/or micrografting, or for micrografting a wound, wound site, a disease lesion, a biofilm, or a surgical site; or, for micrografting a wound, a wound site, a disease lesion, a biofilm or a surgical site or any micrograft application site, for rapid re-epithelialization, or for micrografting a wound, wound site (e.g., a biofilm infected wound or disease lesion), a disease lesion or a surgical site for rapid re-epithelialization of large non-healing wounds,

wherein optionally the wound, wound site or disease lesion is or comprises or is caused by a skin disease wound, a wound site or a skin disease lesion, and optionally the treated or micrografted skin disease wound, wound site or skin disease lesion is or comprises or is caused by a genetic blistering disease, optionally an Epidermolysis Bullosa (EB) or related condition, optionally a simplex EB, a junctional EB, a dystrophic EB (such as a recessive dystrophic epidermolysis bullosa (RDEB)), Kindler syndrome, a revertant EB or a non-revertant EB, or

optionally the treated or micrografted skin disease wound, wound site or skin disease lesion is or comprises or is caused by an infected biofilm, or a drug-resistant-infected drug-resistant, or a biofilm-infected chronic wound of an EB, a revertant EB or a non-revertant EB,

the methods comprising:

(a) providing a product of manufacture, device, or composition as provided herein, or the kit or integrated point of care mixing kit as provided herein, having the mixed thickness skin micrograft, split-thickness skin graft, full thickness skin graft, mixed thickness skin micrograft, skin tissue column, microscopic skin tissue column, “fractional skin harvesting (FSH)” graft, ultra-micrograft, or a microscopic skin tissue column (MSTC),

wherein the sterile hydrogel solution is mixed with the mixed thickness skin micrograft, split-thickness skin graft, or full thickness skin graft/pure water or isotonic solution or buffer suspension within between about 0.5 minutes and 2 hours, or between about 0.5, 1, 2, 3, 4, 5, 6, 7 ,8, 9, 10, 11, 12, 13, 14, 15, 20, 25, 30, 40, 50, 60, 75, 90, 100, 110 or 120 minutes or more of, or just before, application to a micrograft application site or a wound site or surgical site; and

(b) applying the mixture of (a) to a micrograft site, a site prepared for a micrograft, a surgical site, or the wound, wound site, or skin disease site (e.g., EB skin wound or lesion, or an infected biofilm), optionally within between about 0.5 minutes and 2 hours, or between about 0.5, 1, 2, 3, 4, 5, 6, 7 ,8, 9, 10, 11, 12, 13, 14, 15, 20, 25, 30, 40, 50, 60, 75, 90, 100, 110 or 120 minutes or more minutes, of the mixing,

and optionally the mixed thickness skin micrograft, split-thickness skin graft, full thickness skin graft, mixed thickness skin micrograft, skin tissue column, microscopic skin tissue column, “fractional skin harvesting (FSH)” graft, ultra-micrograft, or a microscopic skin tissue column (MSTC) is treated with a collagenase before application to the micrograft site, disease lesion site, surgical site, wound, wound site, or skin disease site (e.g., EB skin wound or lesion, or an infected biofilm), and optionally the collagenase treatment comprises use of about 600U collagenase/ml tissue.

In alternative embodiments, the amount of mixed thickness skin micrograft, split-thickness skin graft, or full thickness skin graft is based on a 10× to 100×, or 5× to 125×, expansion over the micrograft application site, surgical site, wound, wound site, or skin disease site (e.g., EB skin wound or lesion, or an infected biofilm).

In alternative embodiments, the product of manufacture, device, or composition as provided herein, or a mixture as provided herein, is applied to: or the micrograft application site is: (a) a refractory large wound, a wound >10 cm², a chronic wound, a diabetic foot ulcer, a venous leg ulcer, a pressure ulcer, a burn a third degree burn, or a large >10% total body surface area burn or wound; (b) a skin disease wound, a wound site or a skin disease lesion, and optionally the treated or micrografted skin disease wound, wound site or skin disease lesion is or comprises or is caused by a genetic blistering disease, optionally an Epidermolysis Bullosa (EB) or related condition, optionally a simplex EB, a junctional EB, a dystrophic EB, Kindler syndrome, a revertant EB or a non-revertant EB; (c) a sterile carrier, or an implant, wherein optionally the sterile carrier comprises or is a mesh, or a polyester or silicone mesh, or a nonwoven dressing or a porous, non-occlusive dressing; or, (d) a biofilm, or an infected biofilm.

In alternative embodiments, the methods further comprise applying a non-adherent contact layer over all, substantially all or part of the micrograft application site or wound, or skin disease wound, a wound site or a skin disease lesion, or biofilm or infected biofilm, to which the: product of manufacture, device, or composition as provided herein; or, the mixture as provided herein, is applied, wherein optionally the non-adherent contact layer comprises a silicon, a silicon mesh, or a MEPITEL™ (Molnlycke HealthCare, Norcross, GA), wherein optionally the non-adherent contact layer is applied prior to re-epithelialization of the micrograft site or wound, or is applied or reapplied on day 1, day 4, day 7 and day 14; is applied one or more times at a minimum of every other way day or every third day for the first two or three weeks after first applying the: product of manufacture, device, or composition as provided herein; or, a mixture of as provided herein, to the micrograft application site or wound, or skin disease wound, wound site or skin disease lesion, or biofilm or infected biofilm.

In alternative embodiments, the methods further comprise applying above the non-adherent contact layer a combination of:

(a) an antibiotic, a growth factor or an accelerator of cell migration, an antioxidant, or any combination thereof;

(b) a hydrogel, an antibiotic, a growth factor or an accelerator of cell migration, an antioxidant, or any combination thereof;

(c) a hydrogel, an antibiotic, and a growth factor or an accelerator of cell migration;

(d) a hydrogel and an antibiotic;

(e) a hydrogel, an antibiotic, and an antioxidant;

(f) a hydrogel, an antibiotic, a growth factor or an accelerator of cell migration, and an antioxidant, or

(g) any combination of (a) to (f),

wherein optionally the applying of the step (a), (b), (c), (d), (e), (f) or (g), or the any combination of (a) to (g), above the non-adherent contact layer comprises:
(i) applying prior to re-epithelialization of the micrograft site, surgical site, wound, micrograft application site, skin disease wound, wound site or skin disease lesion, or biofilm or infected biofilm,
(ii) applying or re-applying on day 1, day 3, day 7 and day 14, or
(iii) applying one or more times at a minimum of every other way day or every third day for the first two or three weeks after the first applying of the: product of manufacture, device, or composition as provided herein; or, a mixture as provided herein, to the micrograft site, surgical site, wound, micrograft application site, skin disease wound, wound site or skin disease lesion, or biofilm or infected biofilm.

In alternative embodiments of the methods, the antibiotic applied above the non-adherent contact layer comprises an aminoglycoside antibiotic; a gentamicin, a high-dose gentamicin, a gentamicin at a concentration of about 0.1%; a vancomycin, a hygromycin B, a neomycin, a verdamicin; a mutamicin; a sisomicin; a netilmicin; or a retymicin, or any combination thereof (e.g., a gentamicin (e.g., a high-dose gentamicin) and a vancomycin).

In alternative embodiments of the methods, the growth factor applied above the non-adherent contact layer is an erythropoietin, a recombinant erythropoietin, or an epoetin alfa (e.g., PROCRIT™ or EPOGEN™); a granulocyte colony-stimulating factor (G-CSF or GCSF), also known as colony-stimulating factor 3 (CSF 3); a filgrastin or a G-CSF analog, or a FILCAD™ (Cadila Pharmaceuticals), IMUMAX™ (Abbott Laboratories), GRAFEEL™ (Dr. Reddy's Laboratories), NEUKINE (Intas Biopharmaceuticals), EMGRAST™ (Emcure Pharmaceuticals), RELIGRASTTM (Reliance Life Sciences), ZARZIO™ (Sandoz), or a NUFIL™ (Biocon); a keratinocyte growth factor or a palifermin or a KEPIVANCE™ (Biovitrum); or a gamma-aminobutyric acid (GABA); or, any combination thereof.

In alternative embodiments of the methods, the anti-oxidant applied above the non-adherent contact layer comprises a glycyrrhetinic acid (GA) (also known as enoxolone); a deferoxamine (also known as desferrioxamine B, desferoxamine B, DFO, DFO-B, DFOA, DFB or desferal); or a nicotinamide (also known as vitamin B3); a niacin; a vitamin A; a vitamin C; a vitamin E; or any combination thereof, wherein optionally the deferoxamine is at a concentration of about 0.1%, and optionally the nicotinamide is at a concentration of about 1.0%.

In alternative embodiments the methods further comprise applying to the micrograft site, surgical site, wound, micrograft application site, skin disease wound, wound site or skin disease lesion, or biofilm or infected biofilm, after a re-epithelialization and/or after removal of non-adherent contact layer:

(a) an antibiotic, a growth factor, an antioxidant, or any combination thereof;

(b) a hydrogel, an antibiotic, a growth factor, an antioxidant, or any combination thereof;

(c) a hydrogel and an antioxidant;

(d) a hydrogel, a growth factor, and an antioxidant, or

(e) any combination of (a) to (d),

wherein optionally the applying of (a), (b), (c), (d) or (e) is at any one, several or all of between about days 14 through 42, or between about days 7 to 50,

wherein optionally the antibiotic comprises an aminoglycoside antibiotic; a gentamicin, a high-dose gentamicin, a gentamicin at a concentration of about 0.1%; a vancomycin, a hygromycin B, a neomycin, a verdamicin; a mutamicin; a sisomicin; a netilmicin; or a retymicin, or any combination thereof (e.g., a gentamicin (e.g., a high-dose gentamicin) and a vancomycin).

In alternative embodiments of the methods further comprise applying an isotonic solution or buffer to the micrograft site, surgical site, wound, micrograft application site, skin disease wound, wound site or skin disease lesion, or biofilm or infected biofilm, after a re-epithelialization and/or after removal of non-adherent contact layer, or at any one, several or all of days 7 through 42, or days 14 through 30,

wherein optionally the isotonic solution or buffer comprises a saline, a phosphate buffered saline (PBS), or an equivalent buffer,

and optionally the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or

and optionally the PBS is at a concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more.

In alternative embodiments the methods further comprise applying: a nonwoven dressing, an absorbent bandage, a silicone foam dressing, or a MEPILEX™ (Molnlycke HealthCare, Norcross, Ga.).

In alternative embodiments, provided are methods for treating or ameliorating a wound, a micrograft site, a surgical site, a micrograft application site, a skin disease wound, a wound site or a skin disease lesion, or a biofilm or an infected biofilm,

an injury or a tissue or organ defect, or for augmenting or building up of a tissue or organ, a cartilage, a bone structure, a bladder structure, a muscle, or a nerve structure comprising:

(a) providing a product of manufacture, device, or composition as set forth in any of claims 1 to 7 or the kit or integrated point of care mixing kit of claim 8; and

(b) applying the product of manufacture, device, or composition to the wound, micrograft site, surgical site, micrograft application site, skin disease wound, wound site or skin disease lesion, or biofilm or infected biofilm, injury or tissue or organ defect, or the tissue, cartilage or bone structure to be augmented or built up,

wherein optionally the wound or injury is a surgical wound or a surgical resection,

and optionally the tissue (e.g., as in the tissue or organ defect to be treated) is a bone, a cartilage, a tendon, a muscle, a bladder, a nervous tissue, a spinal nerve tissue, a brain tissue, a glial tissue, an eye, a skin tissue, a venous or arterial tissue, a mucosal tissue, a urothelial mucosal tissue, a muscle or heart tissue, a bladder, a brain, a heart, a muscle, a liver, a pancreas, or a urethra, wherein optionally the product of manufacture, device, or the composition further comprises a cell, a dissociated organ, or a tissue, and optionally the tissue is “reverted” EB skin tissue graft,

and optionally the cell is a stem cell, or optionally the cell is derived from a bone, a cartilage, a tendon, a muscle, a bladder, a nervous tissue, a spinal nerve tissue, a brain tissue, an eye, a skin tissue, a venous or arterial tissue, a mucosal tissue, a urothelial mucosal tissue, a muscle or heart tissue, a bladder, a brain, a heart, a muscle, a bone, a tendon, a cartilage, a liver, a pancreas, or a urethra, and optionally the organ is a bladder, a brain, a heart, a muscle, a bone, a tendon, a cartilage, a liver, a pancreas, a urethra or an eye.

In alternative embodiments provided are kits, or integrated point of care mixing kits, comprising

(a) the product of manufacture, device, or composition as provided herein, or the kit or integrated point of care mixing kit as provided herein,

(b) a sterile hydrogel as provided herein, or

(c) a sterile hydrogel/mixed thickness skin micrograft, a split-thickness skin graft, a full thickness skin graft, or a full thickness skin graft mixture as used in or made by any method as provided herein,

wherein optionally the sterile hydrogel solution is: (i) in a substantially liquid form capable of setting, gelling or self-assembling; (ii) a partially assembled or gelled hydrogel; or, (iii) in a set, gelled or self-assembled state; or a substantially set, gelled or self-assembled state.

In alternative embodiments the kits, or the integrated point of care mixing kits, further comprise:

(a) a device for micrografting a mixed thickness skin micrograft, split-thickness skin graft, or full thickness skin graft;

(b) instructions for practicing any of the methods as provided herein; or

(c) any combination of (a) and (b).

In alternative embodiments provided are methods for treating a wound, a chronic wound, or a surgical wound, comprising:

(a) systemic administration of a 1-(5-oxohexyl)-3, 7-dimethylxanthine, or a pentoxifylline or an oxpentifylline, which optionally is a TRENTAL™ (Sanofi), a PENTOX™, a PENTOXIL™ or a FLEXITAL™, and

(b) (i) application or administration of the ingredients of the kit, or integrated point of care mixing kit, as provided herein; or, (ii) application or administration of the product of manufacture, a device, or a composition as provided herein.

In alternative embodiments provided are therapeutic combinations comprising: (a) a product of manufacture, device, or composition as provided herein, or the kit or integrated point of care mixing kit as provided herein, or (b) a kit, or an integrated point of care mixing kit, as provided herein.

In alternative embodiments of the therapeutic combinations, the growth factor is an erythropoietin, a recombinant erythropoietin, or an epoetin alfa (e.g., PROCRIT™ or EPOGEN™).

In alternative embodiments of the therapeutic combinations, the therapeutic combination is used in the treatment, amelioration or healing of: a refractory large wound, a wound >10 cm², a chronic wound, a diabetic foot ulcer, a venous leg ulcer, a pressure ulcer, a burn a third degree burn, or a large >10% total body surface area burn or wound. In alternative embodiments of the therapeutic combinations, the therapeutic combination is used for: micrografting, or for micrografting a wound or surgical site, or for micrografting a wound, a surgical site or any micrograft application site, for rapid re-epithelialization, or for micrografting a wound or surgical site for rapid re-epithelialization of large non-healing wounds.

In alternative embodiments of the therapeutic combinations, the therapeutic combination is used in the treatment, amelioration or healing of a wound, an injury or a tissue or organ defect, or for augmenting or building up of a tissue or organ, a cartilage or a bone structure, wherein optionally the wound or injury is a surgical wound or a surgical resection, and optionally the tissue or organ is a bone, a cartilage, a tendon, a muscle, a bladder, a nervous tissue, a spinal nerve tissue, a brain tissue, an eye, or a skin tissue, wherein optionally the product of manufacture, device, or composition further comprises a cell or a tissue, and optionally the cell is a stem cell, or optionally the cell is derived from a bone, a cartilage, a tendon, a muscle, a bladder, a nervous tissue, a spinal nerve tissue, a brain tissue, an eye, or a skin tissue.

In alternative embodiments provided are uses of: (a) (i) a product of manufacture, device, or composition as provided herein, or (ii) a kit, or an integrated point of care mixing kit, as provided herein, for:

(1) (i) the treatment, amelioration or healing of: a refractory large wound, a wound >10 cm², a chronic wound, a diabetic foot ulcer, a venous leg ulcer, a pressure ulcer, a burn a third degree burn, or a large >10% total body surface area burn or wound,

(ii) the treatment, amelioration or healing of: a micrograft site, surgical site, wound, micrograft application site, skin disease wound, wound site or skin disease lesion, or biofilm or infected biofilm,

wherein optionally the wound, wound site or disease lesion is or comprises or is caused by a skin disease wound, a wound site or a skin disease lesion, and optionally the treated or micrografted skin disease wound, wound site or skin disease lesion is or comprises or is caused by a genetic blistering disease, optionally an Epidermolysis Bullosa (EB) or related condition, optionally a simplex EB, a junctional EB, a dystrophic EB, Kindler syndrome, a revertant EB or a non-revertant EB, or

optionally the treated or micrografted skin disease wound, wound site or skin disease lesion is or comprises or is caused by an infected biofilm, or a drug-resistant-infected drug-resistant, or a biofilm-infected chronic wound of EB, a revertant EB or a non-revertant EB (e.g., a revertant EB skin graft is applied to a non-revertant EB skin lesion); or

(iii) micrografting, or for micrografting a wound or surgical site, or for micrografting a wound, a surgical site or any micrograft application site, for rapid tissue regeneration, for rapid re-epithelialization, or for micrografting a wound or surgical site for rapid re-epithelialization of large non-healing wounds; or

(2) the treatment, amelioration or healing of a wound, an injury or a tissue or organ defect, or for augmenting or building up of a tissue, a cartilage or a bone structure,

wherein optionally the wound or injury is a surgical wound or a surgical resection, and optionally the tissue or organ is a bone, a cartilage, a tendon, a muscle, a bladder, a nervous tissue, a spinal nerve tissue, a brain tissue, an eye, or a skin tissue,

wherein optionally the product of manufacture, device, or composition further comprises a cell or a tissue, and optionally the cell is a stem cell, or optionally the cell is derived from a bone, a cartilage, a tendon, a muscle, a nervous tissue, a spinal nerve tissue, a brain tissue, an eye, or a skin tissue.

In alternative embodiments of the uses as provided herein: the growth factor is an erythropoietin, a recombinant erythropoietin, or an epoetin alfa (e.g., PROCRIT™ or EPOGEN™); a granulocyte colony-stimulating factor (G-CSF or GCSF), also known as colony-stimulating factor 3 (CSF 3); a filgrastin or a G-CSF analog, or a FILCAD™ (Cadila Pharmaceuticals), IMUMAX™ (Abbott Laboratories), GRAFEEL™ (Dr. Reddy's Laboratories), NEUKINE (Intas Biopharmaceuticals), EMGRAST™ (Emcure Pharmaceuticals), RELIGRAST™ (Reliance Life Sciences), ZARZIO™ (Sandoz), or a NUFIL™ (Biocon); a keratinocyte growth factor or a palifermin or a KEPIVANCE™ (Biovitrum); a gamma-aminobutyric acid (GABA); or, any combination thereof.

In alternative embodiments of the uses as provided herein: the anti-oxidant comprises a glycyrrhetinic acid (GA) (also known as enoxolone); a deferoxamine (also known as desferrioxamine B, desferoxamine B, DFO, DFO-B, DFOA, DFB or desferal); or a nicotinamide (also known as vitamin B3); a niacin; a vitamin A; a vitamin C; a vitamin E; or any combination thereof, wherein optionally the deferoxamine is at a concentration of about 0.1%, and optionally the nicotinamide is at a concentration of about 1.0%.

The details of one or more aspects of the invention are set forth in the description below. Other features, objects, and advantages of the invention will be apparent from the description and from the claims.

All publications, patents and patent applications cited herein are hereby expressly incorporated by reference for all purposes.

### DETAILED DESCRIPTION

In alternative embodiments, provided are products of manufacture, devices and compositions comprising hydrogels and mixed thickness skin micrografts, or full or split-thickness skin grafts, contained or mixed in or within a hydrogel. In alternative embodiments, compositions and methods as provided herein are used for the treatment or amelioration of wounds and surgical sites, and include compositions and methods for micrografting, or for micrografting a wound, or for micrografting a wound for rapid re-epithelialization, or for micrografting a wound for rapid re-epithelialization of large non-healing wounds.

### Hydrogel and Hydrogel Materials

In alternative embodiments, the hydrogel or hydrogel material comprises a self-assembling peptide. In alternative embodiments, the hydrogel or hydrogel material comprises a plurality of synthetic peptides characterized by stable B-sheet structure with ionic side-chain interactions after setting, gelling or self-assembling. In alternative embodiments, the hydrogel or hydrogel material comprises a self-assembling peptide comprising: the sequence Ile-Glu-Ile-Lys-Ile-Glu-Ile-Lys-Ile-Glu-Ile-Lys-Ile (IEIK)₃I (SEQ ID NO:3); or, the sequence Lys-Leu-Asp-Leu-Lys-Leu-Asp-Leu-Lys-Leu-Asp-Leu (KLDL)₃ (SEQ ID NO:2); or, a 16-amino acid synthetic peptide (Ac-[RADA]₄-CONH₂), or SEQ ID NO:1, which optionally can be or comprise a PURAMATRIX™ (PuraMatrix™) (BD Biosciences, San Jose, Calif.), a PURASTAT™ (PuraStat™) (BD Biosciences, San Jose, Calif.), or a PURADERJVI™ (PuraDerm™) (3DMatrix, Ltd, Tokyo, Japan), or equivalents.

PURAMATRIX™ (PuraMatrix™) and PURASTAT™ (PuraStat™) comprise a laboratory-designed, 16-amino acid polypeptide with a repeating sequence of arginine, alanine, and aspartic acid, or RADARADARADARADA (termed RADA₄ or [RADA]₄) (SEQ ID NO:1). The alternating positively and negatively charged amino acids (arginine and aspartic acid), along with the non-polar alanines in-between the charged amino acids, create two distinct structural surfaces, one hydrophilic and the other hydrophobic (Zhang and Altman, 1999[5]). The RADA polypeptide monomer building blocks form (3-sheet structures upon exposure to physiological concentrations of salt, i.e., tissue culture media orphysiological fluids such as blood, via complementary ionic bond formation at the hydrophilic surface of the molecules (Hauser, et al. 2010 [3]).

With regard to fibril formation, the hydrophobic sides of the peptide form a double sheet inside of the fibers and the hydrophilic side forms the outside of the nanofibers that interact with water molecules, forming an extremely high water content hydrogel; for example, in one embodiment, a PURASTAT® (PuraStat®) or equivalent hydrogel comprising 2.5% RADA peptide or equivalent and 97.5% water is used to practice the invention.

PURASTAT® (PuraStat®), based on the self-assembling peptide platform technology of PURAMATRIX™ (PuraMatrix™), is a CE (Conformite Européenne, meaning “European Conformity”) mark approved surgical hemostatic agent. PuraStat® safe, synthetic, non-biogenic, biocompatible, resorbable peptide hydrogel with no risk of transmissible spongiform encephalopathy (TSE) transmission. PURASTAT® (PuraStat®), a fully transparent slightly viscous aqueous peptide (2.5%) solution, is sold in a pre-filled syringe and is currently available in 1 mL, 3 mL and 5 mL unit doses indicated for hemostasis in several surgical circumstances.

### Harvesting of Skin Micrografts

In alternative embodiments, mixed thickness skin micrografts, split-thickness skin grafts, or full thickness skin grafts, including autologous grafts, used to practice this invention are harvested and/or prepared, e.g., “minced”, using any device or protocol, e.g., using an XPANSION® device or an XPANSION MICROGRAFTING SYSTEM® (SteadMed Medical, Fort Worth, Tex.), or equivalents.

In alternative embodiments, mixed thickness skin micrografts, split-thickness skin grafts, or full thickness skin grafts used to practice this invention are harvested and/or prepared using any device or technique or protocol, e.g., any device or technique that can mince or perforate a skin autograft, a dermatome, e.g., a HUMECA™ dermatome, a Tanner dermatome, a ZIMMER™ dermatome, a Bioplast dermatome, or any modified or handmade device, including devices and protocols as described e.g., by Hadjiiski, THE METHOD OF MICROGRAFTING IN THE TREATMENT OF LARGE AREA FULL-THICKNESS BURNS, Annals of Burns and Fire Disasters, vol. XIII, n.3, September 2000.

Fractional Skin Harvesting

In alternative embodiments, practicing this invention comprises use of “fractional skin harvesting”, or ultra-micrografts, including use of methods of harvesting grafts, e.g., ultra-micrografts or microscopic skin tissue columns (MSTCs), including autologous grafts, that creates no donor site tissue injury.

In alternative embodiments, fractional skin harvesting (FSH) comprises use of harvesting devices or harvesting needles or equivalents, e.g., including harvesting devices produced by honing standard hypodermic needles to have 2 cutting edges. Different harvesting-needle sizes can be used. In alternative embodiments, devices utilize a hypodermic needle with a specific cutting-geometry to core skin tissue mechanically.

When the needle is inserted through full thickness skin and withdrawn, a column of tissue is extracted. A fluidic device is used (or constructed), in which each extracted column is removed from the harvesting needle by negative pressure, and transported through a tube of flowing air and normal saline into a container or a collection vehicle, e.g., a collection basket.

In alternative embodiments, an advantage of using a punch graft to harvest a graft, e.g., an autologous graft, to practice this invention, e.g., for the treatment of wounds, disease lesions, e.g., chronic wounds as found in EB, including EB with “revertant” skin (see below), is the small size of each single (e.g., autologous) graft. In some applications this can be important because control over the size of the graft is advantageous, for example, to have a small controlled size punch graft, e.g., for treating revertant EB, where the “revertant” patches on the patient's body are often small, multiple in number, and irregular in shape and therefore the small size of punch biopsy specimens gives better control over which area is harvested and maximizes their use.

In alternative embodiments, a tissue core is removed from a donor site into a collecting basket by air and fluid flows. The air flow transports the tissue core, while the fluid flow serves the purpose of lubrication for tissue transport and wetting for tissue preservation. In alternative embodiments, the FSH device operates at 55.16 kPa (8 psi) gauge pressure and 208 ml/min saline flow rate, cored 800 μm diameter×2.5 mm length skin columns using a 1.05/0.81 mm outer/inner diameter needle. The MSTC harvesting rate can be about at 1 column/sec; and for this columns size, about 50 MSTCs are required to cover a 1.5 cm×1.5 cm wound. In comparison to split-thickness skin grafts (STSGs), the exemplary FSG method can provide a healing outcomes on the donor and wound sites where the donor site heals without morbidity by remodeling tissue, as opposed to scarring. STSGs can be prepared by harvesting split-thickness skin tissue using an electric-powered dermatome (e.g., as by Nouvag USA, Lake Hughes, Calif.), e.g., set to a cutting depth of 0.55 mm.

An FSH method, or FSH device, can the capability of extracting full-thickness skin columns while preserving their viability and eliminating the donor site morbidity associated with skin grafting. In alternative embodiments, methods used to practice the invention include those as described in e.g., Franco, et al., J. Med. Devices 8(4):041005 (Aug. 19, 2014); Tam et al., Plast. Reconstr. Surg. Glob. Open 2013 Sep. 7; 1(6):e47, Epub 2013 Oct. 7; June K. Robinson, et al., Surgery of the Skin: Procedural Dermatology, Elsevier Health Sciences, Oct. 20, 2014.

### Application of Skin Micrografts

In alternative embodiments, provided are methods for micrografting, or for micrografting a wound or surgical site, or for micrografting a wound, a surgical site or any micrograft application site, or for grafting or micrografting a disease lesion, or a biofilm for rapid re-epithelialization, or for micrografting a wound or surgical site for rapid re-epithelialization of large non-healing wounds, comprising applying (e.g., to a site prepared for a micrograft, a surgical site, or a wound, e.g., a debrided site) a mixture of a sterile hydrogel solution and a mixed thickness skin micrograft, split-thickness skin graft, or full thickness skin graft, which can be in a pure water or isotonic solution or buffer suspension. This mixture can be applied using any device, e.g., a Double-Cartridge Delivery System or a Double-Syringe Delivery System made by MEDMIX SYSTEMS AG, Rotkreuz, Switzerland), or modifications or equivalents thereof.

In alternative embodiments, the treated or micrografted skin disease wound, wound site or skin disease lesion is or comprises or is caused by a genetic blistering disease, optionally an Epidermolysis Bullosa (EB) or related condition, optionally a simplex EB, a junctional EB, a dystrophic EB, Kindler syndrome, a revertant EB or a non-revertant EB, or the treated or micrografted skin disease wound, wound site or skin disease lesion is or comprises or is caused by an infected biofilm, or a drug-resistant-infected drug-resistant, or a biofilm-infected chronic wound of EB, a revertant EB or a non-revertant EB.

In alternative embodiments, a preferred time of grafting is three to seven days after debridement of a wound. The wound site can be treated with local antimicrobials. If indicated, systemic antibiotics can be used between debridement and grafting. Perioperative systemic antibiotics can be given before grafting and continue for one week postoperatively.

In alternative embodiments, for wound bed preparation, all necrotic material are removed with sharp (surgical) or enzymatic debridement. Complete narrow excision of the wound edges and the base may be preferred when possible to create a clean, granulating bed prior to grafting. This removes necrotic material and biofilm and reduces the bacterial count. Bleeding can be stopped with cautery or silver nitrate sticks. Prior to grafting, the wound can be prepped with an anti-septic.

Autologous “Revertant” Epidermolysis Bullosa (EB)

In alternative embodiments, Epidermolysis Bullosa (EB), a group of genetic blistering diseases, is treated or ameliorated using compositions and/or methods as set forth herein. Because of “revertant mosaicism” in EB cells, where carrying disease-causing mutations coexist in one individual with cells in which the inherited mutation is genetically corrected by a spontaneous genetic event (the so-called “revertant cells”), the naturally corrected, or “revertant” EB cells, or keratinocytes can be used in autologous cell therapy and the methods and compositions as provided herein, for example, “revertant mosaic” EB cells (alone or with “normal” non-EB cells) can be the source of grafts, e.g., autologous grafts, or cells used in compositions and/or methods as provided herein, including compositions and/or methods for treating EB as provided herein. For example, “revertant mosaic” EB cells or tissue, or healthy autologous tissue (e.g., skin graft), is harvested, e.g., by Fractional Skin Harvesting (see above) of microscopic tissue skin columns or by micrografting of healthy tissue, and then transplanted to a diseased or a lesioned areas.

For example, compositions as provided herein can be used in methods as described e.g., in Gostyński, et al., J. Am. Acad. Dermatol. 2014 January; 70(1):98-101, who treated persistent ulcers in a patient with non-Herlitz junctional EB caused by mutations in the LAMB3 gene by transplantation of split-thickness biopsy specimens from one of his revertant patches; and, all transplanted biopsy specimens were accepted and complete re-epithelialization occurred within 14 days. During 18 months of follow-up, the patient never experienced blisters or wounds in the grafted area, nor in the healed donor site. Immunofluorescence and DNA sequencing showed that acceptor sites healed with transplanted revertant keratinocytes; or methods as described in Gostyński, et al., Br J Dermatol. 2009 August; 161(2):444-7, who used adhesive tape stripping to remove epithelial sheets of transduced autologous keratinocytes; Pasmooij et al., J Clin Invest. 2007 May; 117(5):1240-8; Hsu, et al., Am J Clin Dermatol (2014) 15:1-6.

A number of aspects of the invention have been described. Nevertheless, it will be understood that various modifications may be made without departing from the spirit and scope of the invention. Accordingly, other aspects are within the scope of the following claims.

1. A product of manufacture, a device, or a composition, comprising:
(a) a sterile hydrogel comprising a hydrogel material, wherein the hydrogel is:
(i) in a substantially liquid form capable of setting, gelling or self-assembling;
(ii) a partially assembled or gelled hydrogel, in a partially assembled or gelled form; or,
(iii) in a set, gelled or self-assembled state; or a substantially set, gelled or self-assembled state,
and optionally the set, gelled or self-assembled state is in situ; and
(b) (1)
(i) a mixed thickness skin micrograft, a split-thickness skin graft, or a full thickness skin graft,
wherein the mixed thickness skin micrograft, split-thickness skin graft, or full thickness skin graft is dispersed in, or mixed into, or substantially evenly distributed throughout, the sterile hydrogel,
and optionally the mixed thickness skin micrograft, split-thickness skin graft, or full thickness skin graft further comprises, or is dispersed in, or mixed into, or substantially evenly distributed throughout, a sterile pure water or a sterile isotonic solution or buffer, or equivalent,
and optionally the micrograft or skin graft is an autologous micrograft;
(ii) a skin tissue column, a microscopic skin tissue column or a skin graft comprising a full-thickness column of skin tissue, a “fractional skin harvesting (FSH)” graft, an ultra-micrograft, a microscopic skin tissue column (MSTC),
wherein optionally the skin tissue column, the micrograft, FSH or skin graft is an autologous graft,
and optionally the skin tissue column, the micrograft, FSH or skin graft is derived from revertant Epidermolysis Bullosa (EB) skin tissue;
(iii) a cell, a tissue or an organ preparation,
and optionally the graft, skin tissue column, or micrograft is an autologous graft, skin tissue column, or micrograft,
wherein optionally the tissue or organ is partially, substantially or fully dissociated or disrupted, and optionally the partially, substantially or fully dissociated or disrupted tissue or organ is dispersed in, or mixed into, or substantially evenly distributed throughout, the sterile hydrogel, and optionally the partially, substantially or fully dissociated or disrupted tissue or organ is substantially evenly distributed throughout, a sterile pure water or a sterile isotonic solution or buffer, or equivalent,
and optionally the tissue or organ is partially, substantially or fully dissociated or disrupted by an enzymatic treatment or a physical dissociation or disruption,
and optionally the enzymatic treatment comprises a collagenase treatment,
and optionally the cell, tissue or organ preparation comprises a collagenase, and optionally the collagenase treatment comprises use of about 600U collagenase/ml tissue,
and optionally the cell is a stem cell, or optionally the cell is derived from a bone, a cartilage, a tendon, a muscle, a bladder, a nervous tissue, a spinal nerve tissue, a brain tissue, a glial tissue, an eye, a skin tissue, a venous or arterial tissue, a mucosal tissue, a urothelial mucosal tissue, a muscle or heart tissue, a bladder, a brain, a heart, a liver, a pancreas, or a urethra,
and optionally the cell is a stem cell, an undifferentiated cell, a de-differentiated cell, a pluripotent cell, an omnipotent cell, an umbilical cord blood cell, or a tissue culture cell,
and optionally the organ is a bladder, a brain, a heart, a muscle, a bone, a tendon, a cartilage, a liver, a pancreas, a urethra or an eye, or
(iv) the mixed thickness skin micrograft, a split-thickness skin graft, or a full thickness skin graft of (i), the skin tissue column, microscopic skin tissue column or skin graft comprising a full-thickness column of skin tissue of (ii), or the cell, a tissue or an organ preparation of (iii), further comprising an enzyme, wherein optionally the enzyme is a collagenase or a hyaluronidase;
(2) a hemostatic agent, wherein optionally the hemostatic agent comprises a tranexamic acid, or a synthetic analog of the amino acid lysine;
(3) a growth factor or an accelerator of cell migration,
wherein optionally the growth factor is an erythropoietin, a recombinant erythropoietin, or an epoetin alfa (e.g., PROCRIT™ or EPOGEN™); a granulocyte colony-stimulating factor (G-CSF or GCSF), also known as colony-stimulating factor 3 (CSF 3); a filgrastin or a G-CSF analog, or a FILCAD™ (Cadila Pharmaceuticals), IMUMAX™ (Abbott Laboratories), GRAFEEL™ (Dr. Reddy's Laboratories), NEUKINE (Intas Biopharmaceuticals), EMGRAST™ (Emcure Pharmaceuticals), RELIGRAST™ (Reliance Life Sciences), ZARZIO™ (Sandoz), or a NUFIL™ (Biocon); a keratinocyte growth factor or a palifermin or a KEPIVANCE™ (Biovitrum);
a gamma-am inobutyric acid (GABA); or, any combination thereof, wherein optionally the accelerator of cell migration comprises an inhibitor of a microtubule-severing enzyme, an inhibitor of microtubule degradation or an accelerator of microtubule formation, and optionally the microtubule-severing enzyme comprises an fidgetin-like 2 (FL2) enzyme and the inhibitor of a microtubule-severing enzyme comprises an inhibitor of FL2, and optionally the FL2 inhibitor comprises an FL2-inhibiting antisense nucleotide (e.g., an antisense RNA) or an FL2-inhibiting siRNA;
(4) an anti-oxidant,
wherein optionally the anti-oxidant comprises: a glycyrrhetinic acid (GA) (also known as enoxolone), a nicotinamide (also known as vitamin B3), a niacin, a vitamin A, a vitamin C, a vitamin E or any tocopherol or tocotrienol, or a deferoxamine (also known as desferrioxamine B, desferoxamine B, DFO, DFO-B, DFOA, DFB or desferal),
and optionally the deferoxamine is at a concentration of about 0.1%,
and optionally the nicotinamide is at a concentration of about 0.1%;
(5) an aminoglycoside, wherein optionally the aminoglycoside comprises a gentamicin; or
(6) a combination of (1), (2), (3) and (4);
a combination of (2), (3) and (4);
a combination of (2) and (3);
a combination of (3) and (4);
a combination of (2) and (4);
a combination of (1), (2) and (3);
a combination of (1), (2) and (4);
a combination of (1) , (3) and (4);
a combination of (1) and (2);
a combination of (1) and (3);
a combination of (1) and (4);
a combination of (1), (2), (3), (4) and (5);
a combination of (1), and (5);
a combination of (1), (2), and (5);
a combination of (1), (3) and (5);
a combination of (1), (4) and (5);
a combination of (1), (2), (3) and (5);
a combination of (1), (2), (4) and (5);
a combination of (1), (3), (4) and (5); or
any combination of (1), (2), (3), (4) and/or (5).

2. The product of manufacture, device, or composition of claim 1, wherein:
(a) the mixed thickness skin micrograft, split-thickness skin graft, or full thickness skin graft of 1(b) is a minced mixed thickness skin micrograft, split-thickness skin graft, or full thickness skin graft;
(b) the mixed thickness skin micrograft, split-thickness skin graft, or full thickness skin graft has been:
(i) subjected to a mincing procedure,
(ii) substantially cut into a plurality of pieces,
(iii) subjected to a collagenase treatment, and optionally the collagenase treatment comprises use of about 600U collagenase/ml tissue, or
(iv) has been subjected to a mincing procedure or substantially cut into a plurality of pieces and subjected to a collagenase treatment;
and optionally the mixed thickness skin micrograft, split-thickness skin graft, or full thickness skin graft is minced before mixing a sterile pure water or isotonic solution or buffer with the mixed thickness skin micrograft, split-thickness skin graft, or full thickness skin graft,
and optionally the mixed thickness skin micrograft, split-thickness skin graft, or full thickness skin graft is harvested and/or minced using an XPANSION® device or an XPANSION MICROGRAFTING SYSTEM® (SteadMed Medical, Fort Worth, Tex.);
(c) the mixed thickness skin micrograft, split-thickness skin graft, or full thickness skin graft is harvested from a host or donor as a graft of about 0.012″ to 0.016″ in thickness,
wherein optionally the harvested mixed thickness skin micrograft, split-thickness skin graft, or full thickness skin graft is mixed in sufficient pure water, isotonic solution or buffer to result in a suspension,
and optionally the amount of mixed thickness skin micrograft, split-thickness skin graft, or full thickness skin graft is sufficient to substantially cover a donor site tissue area equal to: about ⅓^{rd} to 1/100^{th}, or about 1/10^{th} to 1/100^{th}, of the area of an intended recipient site or the area to receive the graft; or
(d) the mixed thickness skin micrograft, split-thickness skin graft, or full thickness skin graft is a human mixed thickness skin micrograft, split-thickness skin graft, or full thickness skin graft.

3. The product of manufacture, device, or composition of claim 1, wherein:
(a) the hydrogel is capable of self-assembling, gelling or setting when exposed to an environment comprising a salt concentrations ≧1 mM, or gelation, self-assembly or setting is initiated by salt concentrations ≧1 mM;
(b) the hydrogel is capable of self-assembling, gelling or setting into a 3D hydrogel having a nanometer scale and/or a fibrous structure with an average pore size of between about 50 to 200 nm; or
(c) the hydrogel is at a concentration of about: 0.1% to 5% (w/v), 0.5% to 4% (w/v), 1% to 3% (w/v), 1% to 10% (w/v), 1% to 15% (w/v), 1% to 20% (w/v), 1% to 25% (w/v), 1% to 30% (w/v), 1% to 40% (w/v), or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more (w/v); or
(d) the product of manufacture, device or composition of (a), wherein:
(1) the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
(2) the PBS is at a concentration of about 0.25% to 2.5%, 0.25% to 1.5%, 0.5% to 1.0%, 0.54%, 0.6%, 0.7%, 0.8%, 0.9% or 1.0%,or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more.

4. The product of manufacture, device, or composition of claim 1, wherein:
(a) the hydrogel or hydrogel material comprises a self-assembling peptide;
(b) the hydrogel or hydrogel material comprises a plurality of synthetic peptides characterized by stable B-sheet structure with ionic side-chain interactions after setting, gelling or self-assembling;
(c) the hydrogel or hydrogel material comprises a 16-amino acid synthetic peptide (Ac-[RADA]₄-CONH2), or SEQ ID NO:1, and optionally the hydrogel comprises PURAMATRIX™ (PuraMatrix™) (BD Biosciences, San Jose, Calif.), or PURADERM™ (PuraDerm™) (3DMatrix, Ltd, Tokyo, Japan);
(d) the hydrogel or hydrogel material comprises a self-assembling peptide comprising the sequence Lys-Leu-Asp-Leu-Lys-Leu-Asp-Leu-Lys-Leu-Asp-Leu (KLDL)₃ (SEQ ID NO:2);
(e) the hydrogel or hydrogel material comprises a self-assembling peptide comprising the sequence Ile-Glu-Ile-Lys-Ile-Glu-Ile-Lys-Ile-Glu-Ile-Lys-Ile (IEIK)₃I (SEQ ID NO:3);
(f) the hydrogel or hydrogel material comprises a cellulose, a chitin, a chitosan or a deacetylated chitin, a laminin, a collagen, an elastin, a fibrin, a gelatin, an alginic acid, a hyaluronic acid (HA), or a combination thereof,
wherein optionally the HA comprise a thiolated HA or a tyraminated HA,
or optionally the collagen comprises a collagen IV or a collagen I,
or optionally the cellulose comprises a hem icellulose methyl cellulose (MC), a hydroxypropyl cellulose (HPC), a hydroxypropylmethyl cellulose (HPMC), a carboxymethyl cellulose (CMC) or a cellulose-inorganic hybrid hydrogel;
(g) the hydrogel or hydrogel material comprises a polyethylene glycol (PEG), a polyethelene glycol diacrylate (PEGDA), an ethylene glycol dimethacrylate (EGDMA); a cyclodextrin; a p-dioxanone; a hydroxyethyl methacrylate; a poly(methyl methacrylate); a methylene-bis-acrylamide; a poly(acrylic acid); a polyacrylonitrile; a poly(butylene oxide); a polycaprolactone; a poly(ethylene imine); a poly(ethylene oxide); a poly(ethyl methacrylate); a propylene fumarate; a poly(glucosylethyl methacrylate); a poly(hydroxy butyrate); a poly(hydroxyethyl methacrylate); a poly(hydroxypropyl methacrylamide); a poly(lactic acid); a poly(lactic-co-glycolic acid); PNIPAAm, poly(N-isopropyl acrylamide); a poly(N-vinyl pyrrolidone); a poly(propylene oxide); a poly(vinyl alcohol); a poly(vinyl acetate); a poly(vinyl amine), or any combination thereof; or
(h) the hydrogel or hydrogel material comprises any combination of (a) to (g).

5. The product of manufacture, device, or composition of claim 1, wherein
(a) the isotonic solution or buffer comprises a saline, a phosphate buffered saline (PBS), or an equivalent buffer;
(b) the product of manufacture, device or composition of (a), wherein:
(1) the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
(2) the PBS is at a concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
(c) the mixed thickness skin micrograft, split-thickness skin graft, or full thickness skin graft is mixed with sufficient pure water, isotonic solution or buffer to result in a suspension comprising between about a 1.5% to 15% concentration (skin micrograft) per unit weight, or between about a 1.0% to 20% concentration (skin micrograft) per unit weight, or between about a 1.0% to 20% concentration (skin micrograft) per unit volume, or between about a 0.1% to 10% concentration (skin micrograft) per unit volume, of mixed thickness skin micrograft, split-thickness skin graft, or full thickness skin graft in the pure water, isotonic solution or buffer,
and optionally the suspension is then mixed at a ratio of 2 parts suspension to 3 parts hydrogel solution, or 1 to 3 parts suspension to 2 to 4 parts hydrogel solution, to make final product of manufacture or composition having:
about 0.25% to 3.0%, 0.5% to 2.0%, 1%, 1.5%, 2%, 2.5%, 3% or more hydrogel,
about 1% to 10% mixed thickness skin micrograft, split-thickness skin graft, or full thickness skin graft suspension; and/or
about 0.25% to 1.5%, 0.5% to 1.0%, 0.54%, 0.6%, 0.7%, 0.8%, 0.9% or 1.0% or more pure water, isotonic solution or buffer.

6. The product of manufacture, device, or composition of claim 1, wherein the product of manufacture or composition, or the hydrogel, or isotonic solution, comprises:
(a) a tranexamic acid or a synthetic analog of the amino acid lysine;
(b) an erythropoietin, a recombinant erythropoietin, or an epoetin alfa (e.g., PROCRIT™ or EPOGEN™); and
(c) an anti-oxidant,
wherein optionally the anti-oxidant comprises: a glycyrrhetinic acid (GA) (also known as enoxolone), a nicotinamide (also known as vitamin B3), a niacin, a vitamin A, a vitamin C, a vitamin E, or a deferoxamine (also known as desferrioxamine desferoxamine B, DFO, DFO-B, DFOA, DFB or desferal),
and optionally the deferoxamine is at a concentration of about 0.1%,
and optionally the nicotinamide is at a concentration of about 0.1%.

7. The product of manufacture, device, or composition of claim 1, wherein:
(a) the product of manufacture, device or composition is in situ; or
(b) the product of manufacture or device is or comprises a component or part of a medical device.

8. A kit, or an integrated point of care mixing kit, comprising a product of manufacture, a device, or a composition of claim 1.

9. A method for treating and/or micrografting, or for micrografting a wound, wound site, a disease lesion or surgical site; or, for micrografting a wound, a wound site, a disease lesion or a surgical site or any micrograft application site, for rapid re-epithelialization, or for micrografting a wound, wound site, a disease lesion or a surgical site for rapid re-epithelialization of large non-healing wounds,
wherein optionally the wound, wound site or disease lesion is or comprises or is caused by a skin disease wound, a wound site or a skin disease lesion, and optionally the treated or micrografted skin disease wound, wound site or skin disease lesion is or comprises or is caused by a genetic blistering disease, optionally an Epidermolysis Bullosa (EB) or related condition, optionally a simplex EB, a junctional EB, a dystrophic EB, Kindler syndrome, a revertant EB or a non-revertant EB, or
optionally the treated or micrografted skin disease wound, wound site or skin disease lesion is or comprises or is caused by an infected biofilm, or a drug-resistant-infected drug-resistant, or a biofilm-infected chronic wound of EB, a revertant EB or a non-revertant EB,
the methods comprising:
(a) (i) providing a product of manufacture, device, or composition as set forth in claim 1, having a mixed thickness skin micrograft, split-thickness skin graft, or full thickness skin graft,
wherein the sterile hydrogel solution is mixed with the mixed thickness skin micrograft, split-thickness skin graft, or full thickness skin graft/pure water or isotonic solution or buffer suspension within between about 0.5 minutes and 2 hours, or between about 0.5, 1, 2, 3, 4, 5, 6, 7 ,8, 9, 10, 11, 12, 13, 14, 15, 20, 25, 30, 40, 50, 60, 75, 90, 100, 110 or 120 minutes or more of, or just before, application to a micrograft application site or a wound site or surgical site; and
(ii) applying the mixture of (a) to a micrograft site, a site prepared for a micrograft, a surgical site, or a wound, optionally within between about 0.5 minutes and 2 hours, or between about 0.5, 1, 2, 3, 4, 5, 6, 7 ,8, 9, 10, 11, 12, 13, 14, 15, 20, 25, 30, 40, 50, 60, 75, 90, 100, 110 or 120 minutes or more minutes, of the mixing,
and optionally the mixed thickness skin micrograft, split-thickness skin graft, or full thickness skin graft, skin tissue column, microscopic skin tissue column, “fractional skin harvesting (FSH)” graft, ultra-micrograft, or a microscopic skin tissue column (MSTC), is treated with a collagenase before application to the micrograft site, surgical site, wound, wound site, or skin disease site (e.g., EB skin wound or lesion, or an infected biofilm), and optionally the collagenase treatment comprises use of about 600U collagenase/ml tissue;
(b) the method of (a), wherein the amount of mixed thickness skin micrograft, split-thickness skin graft, or full thickness skin graft is based on a 10× to 100×, or 5× to 125×, expansion over the micrograft application site, surgical site, wound, wound site, or skin disease site, or EB skin wound or lesion, or an infected biofilm;
(c) the method of (a) or (b), wherein the product of manufacture, device, or composition or the mixture is applied to: or the micrograft application site is:
(i) a refractory large wound, a wound >10 cm², a chronic wound, a diabetic foot ulcer, a venous leg ulcer, a pressure ulcer, a burn a third degree burn, or a large >10% total body surface area burn or wound;
(ii) a skin disease wound, a wound site or a skin disease lesion, and optionally the treated or micrografted skin disease wound, wound site or skin disease lesion is or comprises or is caused by a genetic blistering disease, optionally an Epidermolysis Bullosa (EB) or related condition, optionally a simplex EB, a junctional EB, a dystrophic EB, Kindler syndrome, a revertant EB or a non-revertant EB;
(iii) a sterile carrier, or an implant; or
(vi) a biofilm, or an infected biofilm
wherein optionally the sterile carrier comprises or is a mesh, or a polyester or silicone mesh, or a nonwoven dressing or a porous, non-occlusive dressing;
(d) the method of any of (a) to (c), further comprising applying a non-adherent contact layer over all, substantially all or part of the micrograft application site or wound, or skin disease wound, a wound site or a skin disease lesion, or biofilm or infected biofilm, to which the product of manufacture, device, or composition or the mixture is applied,
wherein optionally the non-adherent contact layer comprises a silicon, a silicon mesh, or a MEPITEL™ (Molnlycke HealthCare, Norcross, Ga.),
wherein optionally the non-adherent contact layer is applied prior to re-epithelialization of the micrograft site or wound, or is applied or reapplied on day 1, day 4, day 7 and day 14; is applied one or more times at a minimum of every other way day or every third day for the first two or three weeks after first applying the: product of manufacture, device, or composition or the mixture to the micrograft site, surgical site, wound, micrograft application site, skin disease wound, wound site or skin disease lesion, or biofilm or infected biofilm;
(e) the method of (d), further comprising applying above the non-adherent contact layer a combination of:
(1) an antibiotic, a growth factor or an accelerator of cell migration, an antioxidant, or any combination thereof;
(2) a hydrogel, an antibiotic, a growth factor or an accelerator of cell migration, an antioxidant, or any combination thereof;
(3) a hydrogel, an antibiotic, and a growth factor or an accelerator of cell migration;
(4) a hydrogel and an antibiotic;
(5) a hydrogel, an antibiotic, and an antioxidant;
(6) a hydrogel, an antibiotic, a growth factor or an accelerator of cell migration, and an antioxidant, or
(7) any combination of (1) to (6),
wherein optionally the applying of the step (1), (2), (3), (4), (5), (6) or (7), or the any combination of (1) to (6), above the non-adherent contact layer comprises:
(i) applying prior to re-epithelization of the micrograft site, surgical site, wound, micrograft application site, skin disease wound, wound site or skin disease lesion, or biofilm or infected biofilm,
(ii) applying or re-applying on day 1, day 3, day 7 and day 14, or
(iii) applying one or more times at a minimum of every other way day or every third day for the first two or three weeks after the first applying of the: product of manufacture, device, or composition or the mixture to the micrograft site, surgical site, wound, micrograft application site, skin disease wound, wound site or skin disease lesion, or biofilm or infected biofilm,
wherein optionally the growth factor is an erythropoietin, a recombinant erythropoietin, or an epoetin alfa (e.g., PROCRIT™ or EPOGEN™); a granulocyte colony-stimulating factor (G-CSF or GCSF), also known as colony-stimulating factor 3 (CSF 3); a filgrastin or a G-CSF analog, or a FILCAD™ (Cadila Pharmaceuticals), IMUMAX™ (Abbott Laboratories), GRAFEEL™ (Dr. Reddy's Laboratories), NEUKINE (Intas Biopharmaceuticals), EMGRAST™ (Emcure Pharmaceuticals), RELIGRAST™ (Reliance Life Sciences), ZARZIO™ (Sandoz), or a NUFIL™ (Biocon); a keratinocyte growth factor or a palifermin or a KEPIVANCE™ (Biovitrum); a gamma-aminobutyric acid (GABA); or, any combination thereof,
wherein optionally the accelerator of cell migration comprises an inhibitor of a microtubule-severing enzyme, an inhibitor of microtubule degradation or an accelerator of microtubule formation, and optionally the microtubule-severing enzyme comprises an fidgetin-like 2 (FL2) enzyme and the inhibitor of a microtubule-severing enzyme comprises an inhibitor of FL2, and optionally the FL2 inhibitor comprises an FL2-inhibiting antisense nucleotide (e.q., an antisense RNA) or an FL2-inhibiting siRNA;
and optionally the antibiotic applied above the non-adherent contact layer comprises: an aminoglycoside antibiotic; a gentamicin, a high-dose gentamicin, a gentamicin at a concentration of about 0.1%; a vancomycin, a hypromycin B, a neomycin, a verdamicin; a mutamicin; a sisomicin; a netilmicin; a retymicin; a gentamicin; a high-dose gentamicin; a high-dose vancomycin, or any combination thereof;
and optionally the growth factor applied above the non-adherent contact layer is an erythropoietin, a recombinant erythropoietin, or an epoetin alfa (e.g., PROCRIT™ or EPOGEN™); a granulocyte colony-stimulating factor (G-CSF or GCSF), also known as colony-stimulating factor 3 (CSF 3); a filgrastin or a G-CSF analog, or a FILCAD™ (Cadila Pharmaceuticals), IMUMAX™ (Abbott Laboratories), GRAFEEL™ (Dr. Reddy's Laboratories), NEUKINE (Intas Biopharmaceuticals), EMGRAST™ (Emcure Pharmaceuticals), RELIGRAST™ (Reliance Life Sciences), ZARZIO™ (Sandoz), or a NUFIL™ (Biocon); a keratinocyte growth factor or a palifermin or a KEPIVANCE™ (Biovitrum); or a gamma-aminobutyric acid (GABA); or, any combination thereof;
and optionally the anti-oxidant applied above the non-adherent contact layer comprises a glycyrrhetinic acid (GA) (also known as enoxolone); a deferoxamine (also known as desferrioxamine B, desferoxamine B, DFO, DFO-B, DFOA, DFB or desferal); or a nicotinamide (also known as vitamin B3); a niacin; a vitamin A; a vitamin C; a vitamin E; or any combination thereof,
wherein optionally the deferoxamine is at a concentration of about 0.1%
and optionally the nicotinamide is at a concentration of about 1.0%;
(f) the method of any (a) to (e), further comprising applying to the micrograft site, the surgical site, or the wound site, after a re-epithelization and/or after removal of non-adherent contact layer:
(1) an antibiotic, a growth factor, an antioxidant, or any combination thereof;
(2) a hydrogel, an antibiotic, a growth factor, an antioxidant, or any combination thereof;
(3) a hydrogel and an antioxidant;
(4) a hydrogel, a growth factor, and an antioxidant, or
(5) any combination of (1) to (5),
wherein optionally the applying of (1), (2), (3), (4) or (5) is at any one, several or all of between about days 14 through 42, or between about days 7 to 50,
wherein optionally the antibiotic comprises an aminoglycoside antibiotic; a gentamicin, a high-dose gentamicin, a gentamicin at a concentration of about 0.1%;
a vancomycin, a a high-dose vancomycin, a hygromycin B, a neomycin, a verdamicin; a mutamicin; a sisomicin; a netilmicin; or a retymicin, or any combination thereof:
(g) the method of any of (a) to (f), further comprising applying an isotonic solution or buffer to the micrograft site, the surgical site, or the wound site, after a re-epithelization and/or after removal of non-adherent contact layer, or at any one, several or all of days 7 through 42, or days 14 through 30,
wherein optionally the isotonic solution or buffer comprises a saline, a phosphate buffered saline (PBS), or an equivalent buffer,
and optionally the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
and optionally the PBS is at a concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
(h) the method of any of (a) to (q), further comprising applying: a nonwoven dressing, an absorbent bandage, a silicone foam dressing, or a MEPILEX™ (Molnlycke HealthCare, Norcross, Ga.).

10-19. (canceled)

20. A method for treating or ameliorating a wound, an injury or a tissue or organ defect, or for augmenting or building up of a tissue or organ, a cartilage, a bone structure, a bladder structure, a muscle, or a nerve structure comprising:
(a) providing a product of manufacture, device, or composition as set forth in claim 1; and
(b) applying the product of manufacture, device, or composition to the wound, injury or tissue or organ defect, or the tissue, cartilage or bone structure to be augmented or built up,
wherein optionally the wound or injury is a surgical wound or a surgical resection,
and optionally the tissue is a bone, a cartilage, a tendon, a muscle, a bladder, a nervous tissue, a spinal nerve tissue, a brain tissue, a glial tissue, an eye, a skin tissue, a venous or arterial tissue, a mucosal tissue, a urothelial mucosal tissue, a muscle or heart tissue, a bladder, a brain, a heart, a muscle, a liver, a pancreas, or a urethra, wherein optionally the product of manufacture, device, or composition further comprises a cell, a dissociated organ, or a tissue,
and optionally the cell is a stem cell, or optionally the cell is derived from a bone, a cartilage, a tendon, a muscle, a bladder, a nervous tissue, a spinal nerve tissue, a brain tissue, an eye, a skin tissue, a venous or arterial tissue, a mucosal tissue, a urothelial mucosal tissue, a muscle or heart tissue, a bladder, a brain, a heart, a muscle, a bone, a tendon, a cartilage, a liver, a pancreas, or a urethra, and optionally the organ is a bladder, a brain, a heart, a muscle, a bone, a tendon, a cartilage, a liver, a pancreas, a urethra or an eye.

21. A kit, or an integrated point of care mixing kit, comprising
(a) the product of manufacture, device, or composition as set forth in claim 1
wherein optionally the sterile hydrogel solution is: (i) in a substantially liquid form capable of setting, gelling or self-assembling; (ii) a partially assembled or gelled hydrogel; or, (iii) in a set, gelled or self-assembled state; or a substantially set, gelled or self-assembled state; or
(b) the kit, or the integrated point of care mixing kit, further comprising:
(i) a device for micrografting a mixed thickness skin micrograft, split-thickness skin graft, or full thickness skin graft;
(ii) instructions; or
(iii) any combination of (i) and (ii).

22. (canceled)

23. A method for treating a wound, a chronic wound, or a surgical wound, comprising:
(a) systemic administration of a 1-(5-oxohexyl)-3, 7-dimethylxanthine, or a pentoxifylline or an oxpentifylline,
which optionally is a TRENTAL™ (Sanofi), a PENTOX™, a PENTOXIL™ or a FLEXITAL™, and
(b)
(i) application or administration of the ingredients of the kit, or integrated point of care mixing kit, of claim 21.

24. A therapeutic combination comprising:
(a) a product of manufacture, device, or composition of claim 1
(b)
the therapeutic combination of (a), wherein the growth factor is an erythropoietin, a recombinant erythropoietin, or an epoetin alfa (e.g., PROCRIT™ or EPOGEN™);
(c) the therapeutic combination of (a) or (b), wherein the therapeutic combination is used in the treatment, amelioration or healing of: a refractory large wound, a wound >10 cm², a chronic wound, a diabetic foot ulcer, a venous leg ulcer, a pressure ulcer, a burn a third degree burn, or a large >10% total body surface area burn or wound;
(d) the therapeutic combination of any of (a) to (c), wherein the therapeutic combination is used for: micrografting, or for micrografting a wound or surgical site, or for micrografting a wound, a surgical site or any micrograft application site, for rapid re-epithelialization, or for micrografting a wound or surgical site for rapid re-epithelialization of large non-healing wounds; or
(e) the therapeutic combination of any of (a) to (d), wherein the therapeutic combination is used in the treatment, amelioration or healing of a wound, an injury or a tissue or organ defect, or for augmenting or building up of a tissue or organ, a cartilage or a bone structure,
wherein optionally the wound or injury is a surgical wound or a surgical resection, and optionally the tissue or organ is a bone, a cartilage, a tendon, a muscle, a bladder, a nervous tissue, a spinal nerve tissue, a brain tissue, an eye, or a skin tissue,
wherein optionally the product of manufacture, device, or composition further comprises a cell or a tissue, and optionally the cell is a stem cell, or optionally the cell is derived from a bone, a cartilage, a tendon, a muscle, a bladder, a nervous tissue, a spinal nerve tissue, a brain tissue, an eye, or a skin tissue.

25-31. (canceled)


SEQUENCE LISTING (3 sequences)

SEQ ID NO: 1 - protein, 16 aa, artificial sequence
  synthetic peptide
RADARADARADARADA

SEQ ID NO: 2 - protein, 12 aa, artificial sequence
  synthetic peptide
KLDLKLDLKLDL

SEQ ID NO: 3 - protein, 13 aa, artificial sequence
  synthetic peptide
IEIKIEIKIEIKI